(19) United States
(12) Patent Application Publication
(10) Pub. No.: US 2016/0045612 A1
(43) Pub. Date: Feb. 18, 2016

(54) FUNCTIONALIZED NANOPARTICLES FOR MEDICAL TREATMENTS

(71) Applicant: UNIVERSITY OF MASSACHUSETTS, Boston, MA (US)

(72) Inventors: Mingdi Yan, Arlington, MA (US); H. Surangi N. JAYAWARDENA, Lowell, MA (US); Kalana W. JAYAWARDENA, Lowell, MA (US); Xuan CHEN, Lowell, MA (US); Thareendra C. DeZOYSA, Lowell, MA (US)

(21) Appl. No.: 14/780,248

(22) PCT Filed: Feb. 19, 2014
(86) PCT No.: PCT/US14/17008

§ 371 Date: Sep. 25, 2015

Related U.S. Application Data
(60) Provisional application No. 61/766,176, filed on Feb. 19, 2013.
(60) Provisional application No. 61/781,017, filed on Mar. 14, 2013.
(60) Provisional application No. 61/939,075, filed on Feb. 12, 2014.

(51) Int. Cl. A61K 47/48; A61K 31/7036

(52) U.S. Cl. CPC A61K 47/48092; A61K 47/48884; A61K 31/7036

(57) ABSTRACT

A class of functionalized nanoparticles useful in medical treatments is described. The nanoparticles have an attached carbohydrate that is selected on the basis that a cell to be treated ingests as a consequence of the presence of the carbohydrate. The nanoparticles have an attached chemical that if inside the cell is capable of treating the cell (e.g., curing a disease condition in the cell, killing the cell if it is pathogenic, or improving the health of the cell). The nanoparticle carries the chemical preferentially into the cell because the cell will ingest the carbohydrate, and thereby allows the nanoparticle and the chemical into itself.

### CROSS-REFERENCE TO RELATED APPLICATIONS

This application claims priority to and the benefit of co-pending U.S. provisional patent application Ser. No. 61/766,176, filed Feb. 19, 2013, co-pending U.S. provisional patent application Ser. No. 61/781,017, filed Mar. 14, 2013, and co-pending U.S. provisional patent application Ser. No. 61/939,075, filed Feb. 12, 2104, each of which applications is incorporated herein by reference in its entirety.

### STATEMENT REGARDING FEDERALLY FUNDED RESEARCH OR DEVELOPMENT

This invention was made with government support under Grants R01GM080295 and 2R15GM066279 awarded by NIH. The government has certain rights in the invention.

### THE NAMES OF THE PARTIES TO A JOINT RESEARCH AGREEMENT

NOT APPLICABLE.

### INCORPORATION-BY-REFERENCE OF MATERIAL SUBMITTED ON A COMPACT DISC

NOT APPLICABLE.

### FIELD OF THE INVENTION

The invention relates to methods for introducing materials into living organisms in general and particularly to a method that employs nanoparticles.

### BACKGROUND OF THE INVENTION

The numerous antibiotic resistant (AR) pathogenic bacterial strains that are emerging is a pressing global challenge that concerns all. Clinically important pathogens acquire resistance creating a menace to public health and significant burden of cost to treat AR infections. According to 2011 report of International Society of Diseases in America (ISDA) data methicillin resistant Staphylococcus aureus (MRSA) infects approximately 19,000 Americans each year with an attributed mortality rate of 6.5% and an estimated cost for treatment around USD 29,000 for each patient.

Resistance development in bacteria was earlier attributed mainly to intrinsic chromosomal mutations and acquisition of elements of resistant DNA from other bacteria. Efflux pumps that expel antibiotic from the cell sprung up from intrinsic chromosomal mutations is now thought of as a major contributor to intrinsic resistance.

Escherichia coli (E. coli) is a food born bacteria, which when pathogenic causes gastrointestinal infections including meningitis and urinary tract infections. E. coli is frequently used as a reliable indicator to detect monitor AR development in fecal bacteria. E. coli developed a staggering 30% increase in streptomycin (Str) resistance from 1950s to 2002.

Some of the problems associated with administering drugs to treat disease in an animal include the possibility that a drug that is toxic to a bacterium or a diseased cell of the animal will also be toxic to a healthy cell of the animal, that the drug will not be specific to the bacterium or to the diseased cells of the animal, and that the drug is expensive. Therefore one would like to be able to direct the drug only to the bacterium or to the diseased cells so as to prevent harming the healthy cells, and so as to avoid having to administer large doses of the drug in order to get sufficient amounts of the drug to the bacterium or the diseased cell, and to minimize the amount of the drug needed to be administered to hold down the cost of the drug.

There is a need for improved methods of administering drugs or other treatments so as to target only the bacteria or diseased cells to be treated.

### SUMMARY OF THE INVENTION

According to one aspect, the invention features a functionalized nanoparticle. The functionalized nanoparticle comprises a nanoparticle having a dimension in the range of units of nanometers to one hundred nanometers, and having an external surface; a carbohydrate attached to the external surface of the nanoparticle, the carbohydrate having a chemical property that can induce a living cell to ingest the functionalized nanoparticle; and a chemical attached to the external surface of the nanoparticle, the chemical known to have a biological effect on the living cell.

In one embodiment, the nanoparticle is selected from the group of nanoparticles consisting of magnetic nanoparticles, silica-coated magnetic nanoparticles, silica nanoparticles, fluorescent silica nanoparticles, and silica-coated quantum dots.

In another embodiment, the carbohydrate is selected from the group of carbohydrate consisting of D-trehalose, D-glucose, D-maltoheptaose, fructose, sucrose, and cyclodextran.

In yet another embodiment, the living cell is selected from the group of cells consisting of a bacterium, a protozoan, a fungus, and a mammalian cell.

In still another embodiment, the biological effect is selected from the group of biological effects consisting of improving the health of a living cell, curing a diseased living cell and killing a diseased living cell.

In a further embodiment, the chemical is an antibiotic.

In still another embodiment, the antibiotic is streptomycin.

In yet another embodiment, the antibiotic is a selected one of ampicillin, vancomycin, kanamycin, isoniazid, rifampicin, amikacin, rifabutin, ethambutol, capreomycin and pyrazinamide.

In an additional embodiment, the antibiotic is a selected one of aminoglycosides, glycopeptides, macrolides, sulfonamides, statins, tetracyclines, peptide antibiotics, antimicrobial peptide, and quinolones.

In one more embodiment, the chemical is a cell penetrating peptide or a therapeutic siRNA.

According to another aspect, the invention relates to a functionalized nanoparticle. The functionalized nanoparticle comprises a nanoparticle having a dimension in the range of units of nanometers to one hundred nanometers, and having an internal surface and an external surface; a carbohydrate attached to the external surface of the nanoparticle, the carbohydrate having a chemical property that can induce a living cell to ingest the functionalized nanoparticle; and a chemical attached to the internal surface of the nanoparticle, the chemical known to have a biological effect on the living cell.

The foregoing and other objects, aspects, features, and advantages of the invention will become more apparent from the following description and from the claims.

### BRIEF DESCRIPTION OF THE DRAWINGS

The objects and features of the invention can be better understood with reference to the drawings described below, and the claims. The drawings are not necessarily to scale, emphasis instead generally being placed upon illustrating the principles of the invention. In the drawings, like numerals are used to indicate like parts throughout the various views.

FIG. 1A is a TEM image of magnetic nanoparticles (MNPs), in which the scale bar represents 20 nm.

FIG. 1B is a TEM image of silica-coated magnetic nanoparticles (SMNPs), in which the scale bar represents 20 nm.

FIG. 1C is a TEM image of Glc-SMNPs, in which the scale bar represents 20 nm.

FIG. 1D is a graph of the DLS size distribution of MNPs in water.

FIG. 1E is a graph of the FT-IR spectrum of PFPA-phosphate functionalized SMNPs.

FIG. 1F is a schematic illustration of the synthesis of MNPs.

FIG. 1G is a schematic illustration of the synthesis of SMNPs.

FIG. 1H is a schematic illustration of the functionalization with PFPA and conjugation of carbohydrates to SMNPs.

FIG. 2A is an optical image of Prussian blue stained A549 cells incubated with Glc-SMNPs.

FIG. 2B is an optical image of Fru-SMNPs.

FIG. 2C is an optical image of Suc-SMNPs.

FIG. 2D is an optical image of SMNPs.

FIG. 3A is a TEM image of A549 cell thin sections after incubating with Glc-SMNPs. Scale bars: 100 nm.

FIG. 3B is another TEM image of A549 cell thin sections after incubating with Glc-SMNPs. Scale bars: 100 nm.

FIG. 4A is an optical image of Prussian blue stained A549 cells incubated in cell growth medium with Glc-SMNPs.

FIG. 4B is an optical image of Fru-SMNPs.

FIG. 4C is an optical image of Suc-SMNPs.

FIG. 4D is an optical image of SMNPs.

FIG. 5A is an optical image of Prussian blue stained primary cells incubated in cell growth medium with Glc-SMNPs.

FIG. 5B is an optical image of Fru-SMNPs.

FIG. 5C is an optical image of Suc-SMNPs.

FIG. 5D is an optical image of SMNPs.

FIG. 6 is a diagram illustrating the synthesis of streptomycin conjugated SNPs (SNP-Str).

FIG. 7A is a diagram illustrating the synthesis of SMNPs and silica-coated quantum dots (SQDs).

FIG. 7B is a diagram illustrating the conjugation of carbohydrates to SQDs, SMNPs, silica nanoparticles (SNPs).

FIG. 7C is a diagram illustrating the conjugation of carbohydrates to MNPs.

FIG. 8A is a diagram illustrating the synthesis of SMNPs.

FIG. 8B is a diagram illustrating the synthesis of PFPA-functionalized SMNPs and conjugation of carbohydrates to SMNPs

FIG. 8C is a diagram illustrating the conjugation of carbohydrates to SNPs, and fluorescent silica nanoparticles (FSNPs).

FIG. 8D is a diagram illustrating the structural formulas of carbohydrates used in conjugation: D-trehalose, D-glucose, D-maltoheptaose and cyclo dextran (CD).

FIG. 9 is a graph showing the comparative difference of minimal inhibitory concentration (MIC) values between streptomycin (Str) and conjugated Str on SNP80-Str, SNP50-Str and SNP30-Str against a streptomycin-resistance strain of E coli.

FIG. 10 is a diagram illustrating the synthesis of maltoheptaose-azide.

FIG. 11 is a diagram illustrating the synthesis of acetylene-terminated PLA.

FIG. 12 is a diagram illustrating the synthesis of maltoheptaose-PLA.

### DETAILED DESCRIPTION

A class of functionalized nanoparticles useful in medical treatments is described. The nanoparticles have an attached carbohydrate that is selected on the basis that a cell to be treated ingests as a consequence of the presence of the carbohydrate. The nanoparticles have an attached chemical that if inside the cell is capable of treating the cell (e.g., curing a disease condition in the cell, killing the cell if it is pathogenic, or improving the health of the cell). The nanoparticle carries the chemical preferentially into the cell because the cell will ingest the carbohydrate, and thereby allows the nanoparticle and the chemical into itself

### Synthesis of Magnetic Nanoparticles (MNPs) and Silane-Coated MNPs (SMNPs)

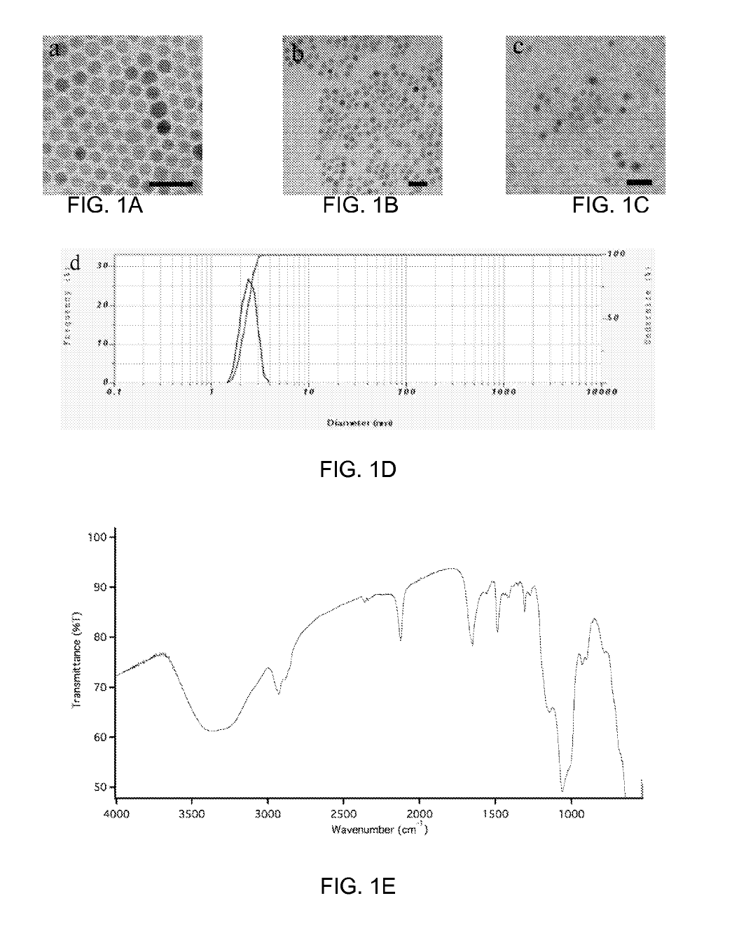

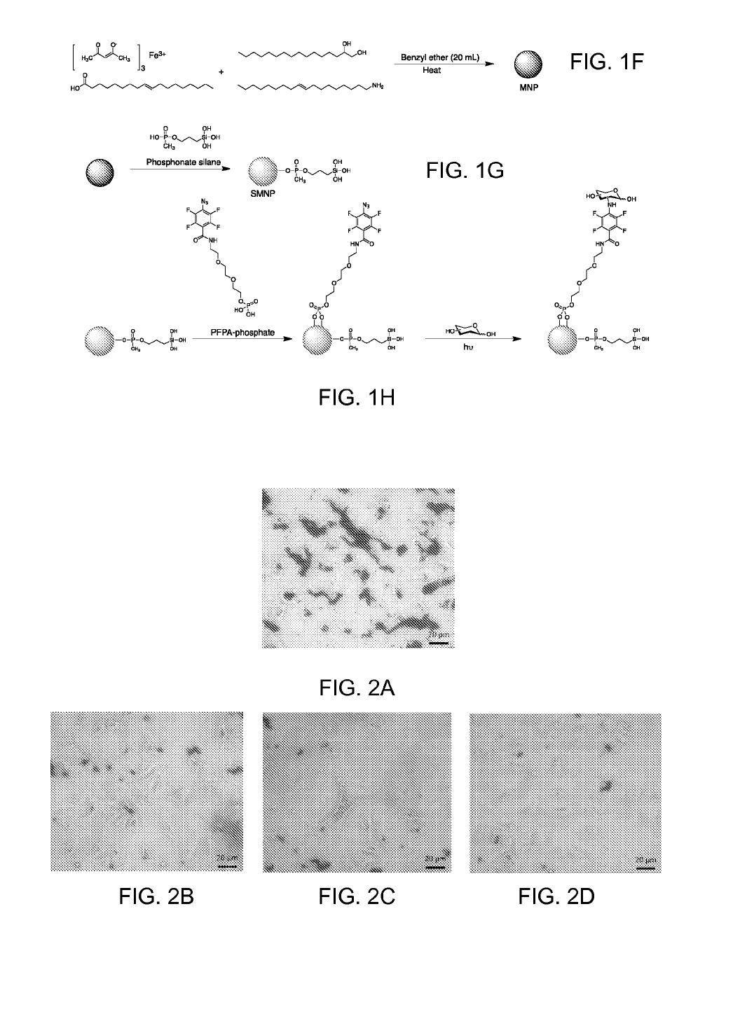

The synthesis of MNPs followed the previously reported procedure of Sun et al. (FIG. 1F).¹ Iron (III) acetylacetonate (0.706 g, 2.0 mmol), 1,2-hexadecanediol (2.584 g, 10.0 mmol), oleic acid (2.239 mL, 6.0 mmol) and oleylamine (2.820 mL, 6 mmol) in dibenzyl ether (20 mL) were stirred under a blanket of nitrogen. The mixture was then heated to 200° C. for 2 h, followed by heating at 300° C. for 1 h. After cooling down to room temperature, ethanol (200-proof, 40 mL) was added and the mixture was centrifuged at 7000 rpm for 10 min. The black precipitate was re-dispersed in hexane (30 mL) containing oleic acid (0.05 mL) and oleylamine (0.05 mL), and the mixture was centrifuged at 6000 rpm for 10 min. The precipitate was discarded, and the supernatant was collected and ethanol was added. After centrifugation, the precipitate was re-dispersed in hexane.

To introduce silane coating on MNPs, a previously reported procedure of Ying et al. was followed.² MNPs (30 mg) were dispersed in toluene (15 mL), and a solution of 3-tri-hydroxysilylpropyl methyl phosphonate sodium salt (phosphonate-silane) in methanol (3 mL, 0.01 M) and a solution of tetramethylammonium hydroxide (TMAH) in methanol (3 mL, 0.01 M) were added, and the mixture was stirred at 80° C. for 3 hrs (FIG. 1F). After cooling down to room temperature, the mixture was centrifuged at 12000 rpm for 30 min and the solid precipitate was re-dispersed in ethanol. This step was repeated for 3 times, and finally the nanoparticles were dispersed in ethanol (15 mL).

### Synthesis of PFPA-Functionalized SMNPs

2-(2′-(2′-(4-Azido-2,3,5,6-tetrafluorobenzamido)ethoxy)-ethoxy)ethyl dihydrogen phosphate (PFPA-phosphate) was synthesized following a previously reported procedure.³ A solution of SMNP (15 mL, 2 mg/mL) was added to a solution of PFPA-phosphate in CHCl₃ (3 mL, 12 mg/mL), and the mixture was stirred at room temperature overnight, and then centrifuged at 12,000 rpm for 30 min. The supernatant was discarded and the pellet was redispersed in acetone. This washing/centrifugation cycle was repeated for 5 times. After the final centrifugation, the supernatant was discarded and the pellet was redispersed in distilled water.

### Conjugation of Carbohydrates to SMNPs

To a glass bottle containing an aqueous solution of SMNPs (1 mL, 1 mg/mL) an aqueous solution of carbohydrate (glucose (Glc), fructose (Fm), or sucrose (Suc) (180 μL, 10 mg/mL)) was added. A 280 nm long-pass optical filter was placed on top of the glass bottle, and the mixture was irradiated with the 450 W medium-pressure Hg lamp (3.2 mW/cm²) for 40 min while stirring. The resulting carbohydrate-conjugated nanoparticles (Glc-SMNPs, Fm-SMNPs, Suc-SMNPs) were purified by centrifugation and re-dispersion in Milli-Q water. Further purification was done by overnight dialysis and finally redispersed in phosphate buffered saline (PBS).

Incubation of Carbohydrate-Conjugated MNPS with A549 Cells

A549 lung carcinoma cell suspensions (10⁶ cells/mL) were prepared in F12K media supplement with 10% fetal bovine serum. From this, 125,000 cells/well were seeded into the 6-well plate and was incubated at 37° C. for 12 h. After the cells were attached to the bottom of the well plate, the media was removed and the cells were repeatedly washed with PBS buffer. Finally, PBS (800 μL) and glyconanoparticles (Glc-SMNPs, Fru-SMNPs, Suc-SMNPs) as well as SMNPs (200 μL, 1 mg/mL) were added to each well, and the cells were incubated at 37° C. for 3 h.

### Prussian Blue Assay

After the cells were incubated with the nanoparticles, the supernatant was removed and the cells were washed with PBS for three times. The cells were then treated with aqueous formaldehyde solution (10%, 0.5 mL) for 30 min to fix the cells, and the wells were washed with PBS for three times. The Prussian blue staining was performed by adding to each well K₄[Fe(CN)₆]•3H₂O (4%, 0.5 mL) and HCl (2%, 0.5 mL), and incubating the cells for at 37° C. for 3 h.

The Prussian blue assay tests the presence of iron. The reaction occurs when treating the iron oxide nanoparticles in the acid solution of ferrocyanide. The ferric ion (Fe(III)) in the iron oxide nanoparticles combines with the ferrocyanide and results in the formation of a bright blue pigment called “Prussian blue” or ferric ferrocyanide. Images in FIG. 2 show that the cells treated with Glc-SMNPs had the most Prussian blue stain in comparison to those treated with Fru-SMNPs, Suc-SMNPs, SMNPs. The results demonstrate that Glc indeed promotes the uptake of the nanoparticles by cells.

### TEM Thin Section Sample Preparation

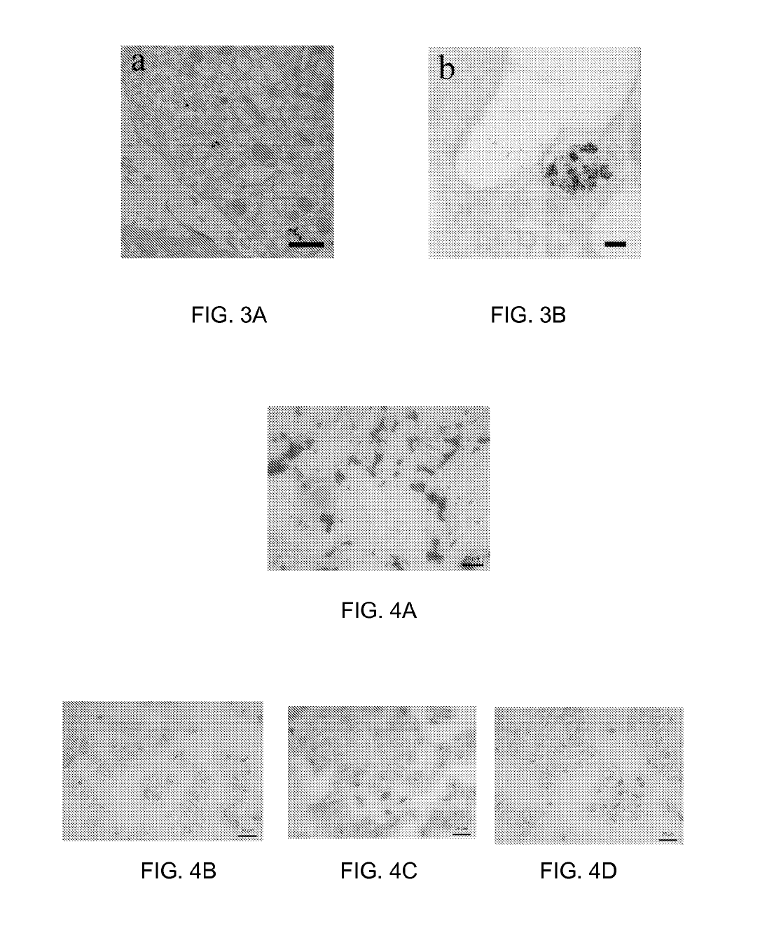

A suspension of nanoparticle-treated A549 cancer cells or untreated A549 cancer cells (1 mL) was centrifuged at 2000 rpm for 7 min, and the supernatant was removed. The pellet was then immersed in a solution of glutaraldehyde in PBS (1.00 mL, 2.5%) in an Eppendorf tube at 4° C. overnight. The glutaraldehyde-fixed pellet was washed 3 times in PBS by incubating the pellet in PBS (1 mL) at 4° C. for 10 min. The pellet was then incubated in a solution of OsO₄ in PBS (2%) at 4° C. for 30 min. The excess OsO₄ was washed with PBS for 3 times by incubating the pellet in PBS (1 mL) at 4° C. for 10 min and removing the supernatant. The pellet was then dehydrated by incubating the pellet in 1 mL of 25%, 30%, 50% and 75% ethanol at 4° C. for 10 min each, followed by 95% ethanol (twice) and 100% ethanol (3 times) at room temperature for 10 min each. Finally, the pellet was incubated in 1 mL of propylene oxide (twice) at room temperature for 10 min each. The embedding resin medium was prepared by mixing Embed-812 (5.0 mL), Araldite 502 (heated to 60° C., 3.0 mL), DDSA (heated to 60 ° C., 11.0 mL) and DMP-30 (0.50 mL), and cooling to room temperature. The pellet was then infiltrated sequentially in propylene oxide/resin (3:1, 2:1, 1:1, 1:2 vol/vol) at room temperature for 15 min each, and finally in the embedding resin. A portion of the pellet with embedding resin was transferred to a BEEM capsule and was allowed to cure overnight at 60° C. in a vacuum oven. The BEEM capsule was then removed and the resin-embedded pellet was cut with an ultramicrotome (Leica EM UC6, Buffalo Grove, Ill.) into ˜100 nm thick slices which were then placed on 200-mesh Cu grids for TEM imaging. TEM thin section images in FIG. 3A and FIG. 3B show that the nanoparticles were indeed internalized by the cells.

Interaction of Primary Cells with Carbohydrate-Conjugated MNPS in Cell Growth Media

Airway epithelial cell suspensions (10⁶ cells/mL) were prepared in primary cell airway epithelial media supplement with small airway epithelial cell growth kit. From this, 125,000 cells/well were seeded into the 6-well plate and was incubated at 37° C. for 12 h. After the cells were attached to the bottom of the well plate, the media was removed and the cells were repeatedly washed with PBS buffer. Finally, airway epithelial media (800 μL) and glyconanoparticles (Glc-SMNPs, Man-SMNPs, Fru-SMNPs, Suc-SMNPs) as well as SMNPs (200 μL, 1 mg/mL) were added to each well, and the cells were incubated at 37° C. for 3 h.

### Prussian Blue Assay

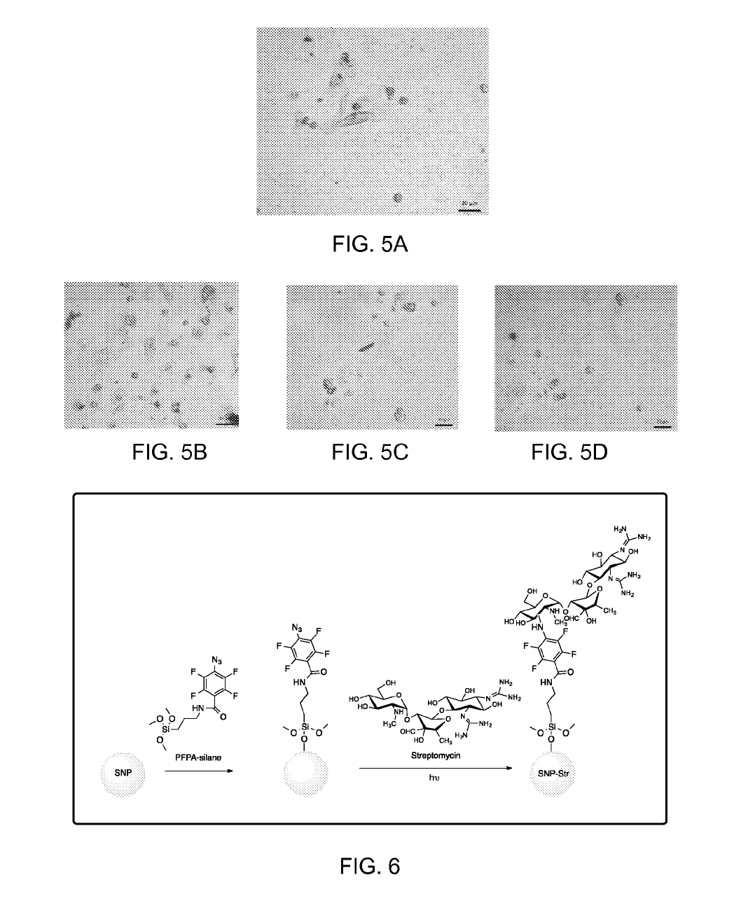

After the cells were incubated with the nanoparticles, the supernatant was removed and the cells were washed with PBS for three times. The cells were then treated with aqueous formaldehyde solution (10%, 0.5 mL) for 30 min to fix the cells, and the wells were washed with PBS for three times. The Prussian blue staining was performed by adding to each well K₄[Fe(CN)₆]•3H₂O (4%, 0.5 mL) and HCl (2%, 0.5 mL), and incubating the cells for at 37° C. for 3 h. The results are shown in FIG. 5A, FIG. 5B, FIG. 5C and FIG. 5D.

Compared to the results in FIG. 4A, FIG. 4B, FIG. 4C and FIG. 4D the amount of Glc-conjugated iron oxide nanoparticle uptake by the primary cells are much less than that by the lung cancer cells. These results demonstrate that Glc promotes the uptake of the nanoparticles by lung cancer cells rather than the primary healthy cells. This is significant that we can use Glc-modified nanoparticles to target cancer cells while minimizing the impact on healthy cells.

### Silica Nanoparticles

Silica nanoparticles were synthesized according to previously developed modified Stober protocol.²² SNP80 was developed by stirring TEOS (2.8 mL) with 6.25% NH3 (2.8 mL) in anhydrous ethanol (34 mL) for 24 h. While SNP50 and SNP30 was prepared by stirring TEOS (2.2 mL or 1.8 mL) with 6.25% NH₃ (2.2 mL or 1.8 mL) in anhydrous ethanol (34 mL) for 48 h. The corresponding yield was obtained for the respective particles SNP80, SNP50, SNP30: 29.8±0.2, 19.7±0.8, 11.7±0.5 mg/mL. To prepare PFPA functionalized SNPs, SNP80, SNP50 and SNP30 were stirred with PFPA-silane (12.6 mM in toluene) respectively in 13.4 mL, 15.8 mL and 19.5 mL for 48 h and refluxed at 76° C. for 1 h (FIG. 6). The resultant particles were centrifuged at 12,000 rpm for 40 min and re-dispersed in acetone this was repeated 4× to remove unreacted precursors and stored in acetone at 4° C. The resultant PFPA conjugated SNP80, SNP50 and SNP30 particles were characterized using DLS and TEM. The DLS measurements for PFPA-silane conjugated SNP80, SNP50 and SNP30 were 81.3±2.2, 43.7±1.3 and 32.9±2.1 nm, while the TEM measurements were 75.3±8.8, 48.1±5.1 and 26.9±4.2 nm.

Streptomycin conjugated SNPs (SNP80-Str, SNP50-Str, SNP30-Str) were prepared by photocoupling PFPA functionalized SNP80, SNP50 and SNP30 (˜1 mg/mL, 2 mL) nanoparticles with 600 μL of 10 mg/mL of aqueous solution of Streptomycin sulfate under UV for 30 min (FIG. 6). The SNP80-Str, SNP50-Str and SNP30-Str were washed in sterile water for 6 times and dialyzed overnight. Particles were checked for sterility by spreading 50 μL on a MH agar plate. The particle diameter determined for SNP80-Str, SNP50-Str and SNP30-Str from DLS were 147.7±2.0, 124.1±2.3 and 100.5±1.8 nm. The same were characterized under TEM sizes respectively for SNP80-Str, SNP50-Str and SNP30-Str were 77.8±13.8, 49.7±5.1 and 30.16±4.8 nm. The disparity in the sizes of the SNP-Str's from DLS and TEM would be due to the presence of agglomeration detected by DLS. The SNP-Str's were characterized by FT-IR to confirm the presence of conjugated streptomycin. Zeta potential characterization of SNP80-Str, SNP50-Str and SNP30-Str were −60.4±1.3, −55.5±2.2 and −80.5±2.6 mV.

SNP-Sty's demonstrate a better antibacterial activity than the free streptomycin when run against an engineered highly streptomycin resistant E. coli. The elevated MIC of 2000 μg/mL of free streptomycin was brought down to as low as 2 log folds by the use of SNP-Str's. Even though not a clinical strain this highly resistant strain could be used successfully to demonstrate the ability of antibiotic nanomaterial conjugates to increase the antibacterial activity of the free drug. The more effective of the 3 types of SNP-Str was the SNP30-Str, its effectiveness mainly attributed to its reduced size. We hypothesize that the greater the amount of particles internalized greater the exerted antibacterial effect. This demonstrates that nanomaterial still hold promise in to increase the effectiveness of resistance formed antibiotics.

### D-Maltoheptaose (G7)

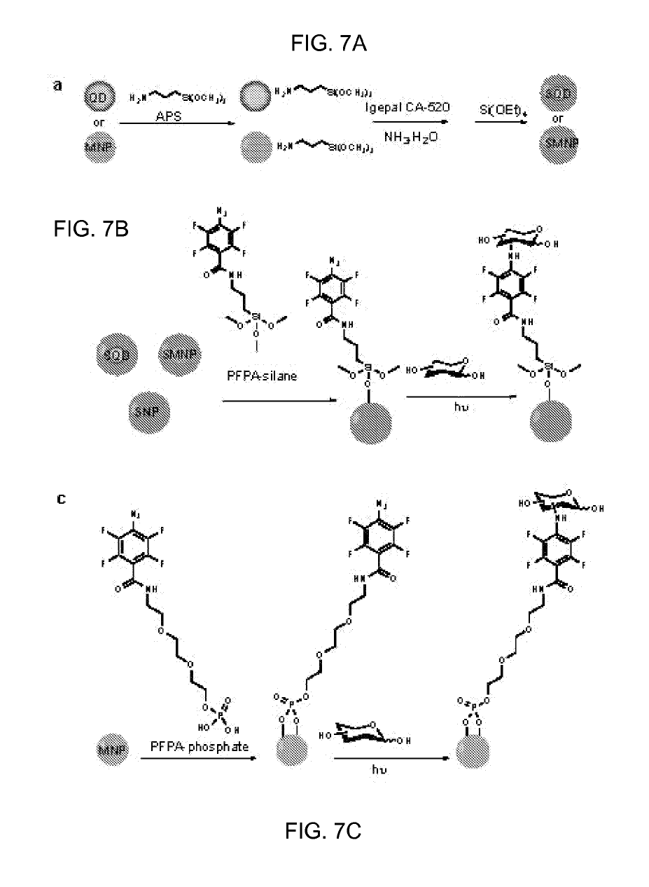

We report that nanoparticles conjugated with D-maltoheptaose (See FIG. 7A, FIG. 7B and FIG. 7C.) showed a striking increase in the surface adherence and internalization by Escherichia coli. This applies to silica nanoparticles, magnetic nanoparticles, silica-coated magnetic nanoparticles and quantum dots ranging from a few to over a hundred nanometers in size, and to E coli strains with or without the maltodextrin transport channel. Four strains of E. coli were used: ATCC 33456, JW3392-1, ORN 178 and ORN 208. Under the same experimental conditions, minimal adhesion or internalization was observed for unfunctionalized or β-cyclodextrin-functionalized nanoparticles. Nanoparticles functionalized with D-mannose (Man) showed the typical surface adhesion on the pili of ORN 178 by way of the Manbinding lectin FimH.

G7, a member of the maltodextrin family that contains 7 glucose units through the α1→4 linkage, was used to test the hypothesis that maltodextrin would increase the uptake and internalization of nanoparticles. Thus, G7-conjugated NPs (G7-SMNPs, G7-SQDs, G7-SNPs) were incubated at 37° C. for 2 h with E. coli strain ATCC 33456 that was harvested at 0.5 OD600. After excess nanoparticles were removed from the medium, the sample was examined by TEM. Unlike Man-conjugated NPs which were attached to the pili of ORN 178, NPs functionalized with G7 adhered to the surface of ATCC 33456 and gained entry into the bacteria.

G7-NPs were treated with JW3392-1. Surface adhesion and subsequent cell wall crossing and internalization were observed for all G7-functionalized nanoparticles. The experiment was then repeated on ORN 178 and ORN 208. Similar to ATCC 33456 and JW3392-1, G7-NPs were uptaken by ORN 178 as well as ORN 208. These results failed to support a mechanism involving the maltodextrin transporter. To further confirm that the nanoparticles were inside the bacteria cells, thin section samples of ATCC 33456 treated with G7-MNPs were prepared. Results showed the presence of G7-MNPs inside the cytoplasm as well as throughout the cell walls.

### Trehalose

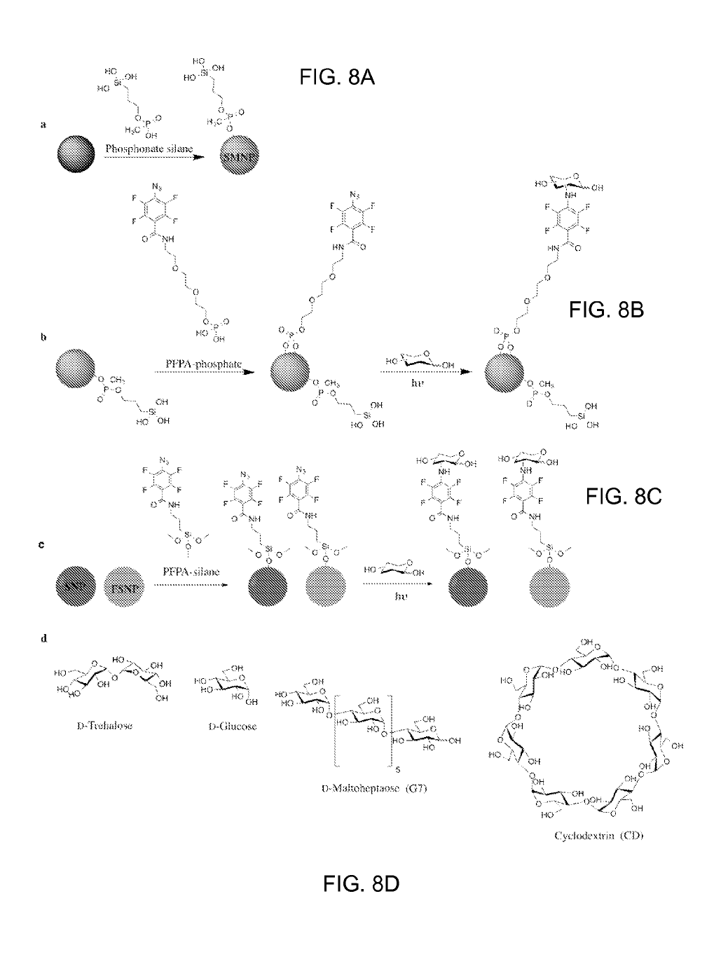

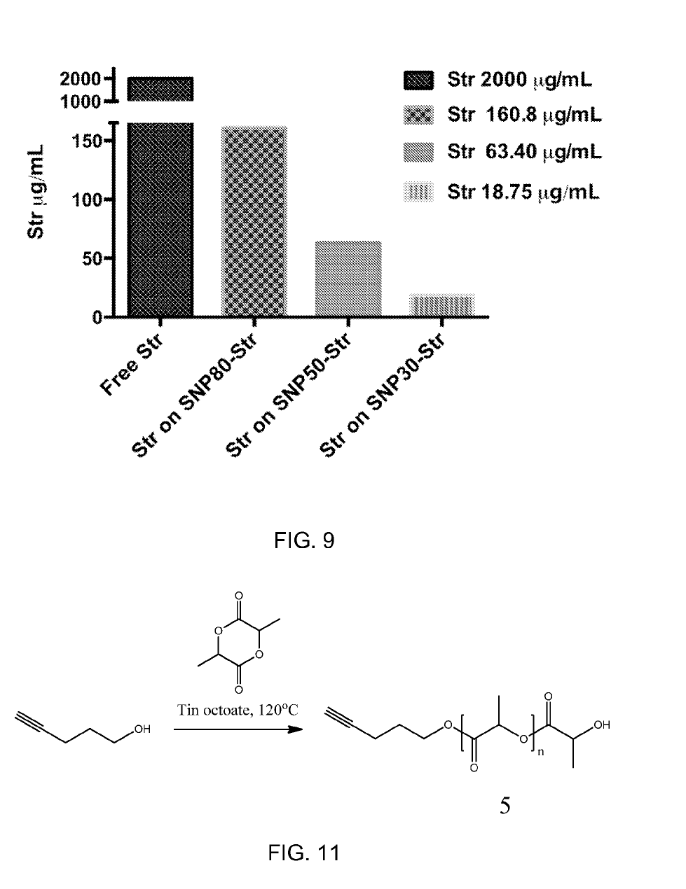

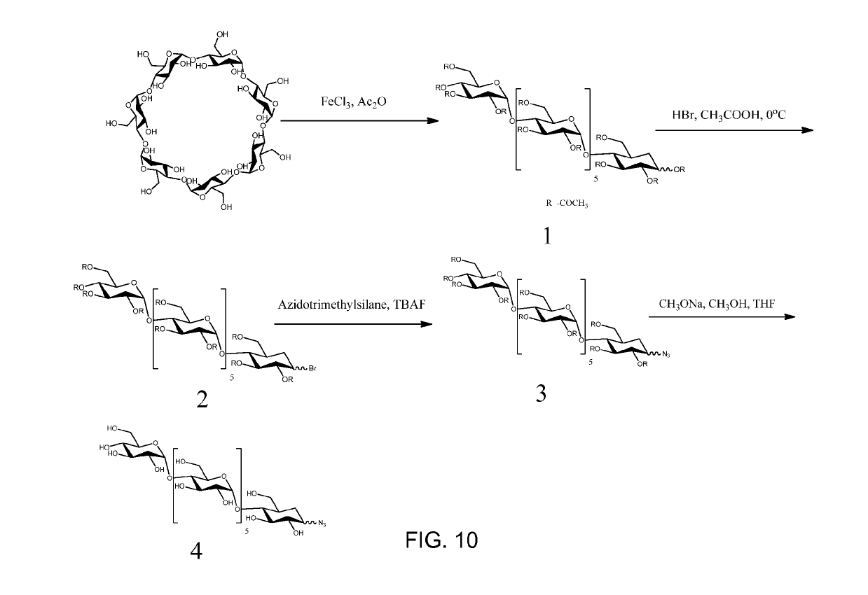

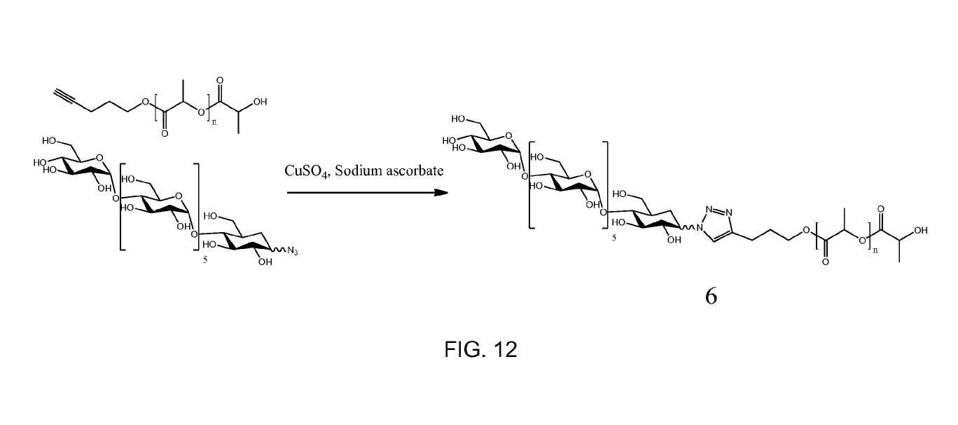

We report that nanoparticles conjugated with trehalose (Tre) (See FIG. 8A, FIG. 8B and FIG. 8C) showed increase in Mycobacterium smegmatis surface adherence and internalization. This applies to nanoparticles ranging from 5 nm to 50 nm. Under the same experimental conditions nanoparticles functionalized with Tre showed surface adherence on infected Mycobacterium smegmatis but showed no surface adherence in type II alveolar epithelial cells (A549) and under same experimental conditions minimum surface adherence internalization was observed unfunctionalized or nanoparticles functionalized with β-cyclodextrin and maltoheptaose.

We report that trehalose promotes the uptake of nanoparticles by M. smegmatis. In addition, the uptake is selective towards bacterial cells, and no enhancement was observed for mammalian cells.

M smegmatis was used because of its close relation to the pathogenic M. tuberculosis. In addition, M. smegmatis is non-pathogenic, and its growth rate is much faster compared to M tuberculosis. M smegmatis is therefore widely used as a common acceptable model for the development of therapeutic drugs against tuberculosis.

Trehalose can be used as an effective ligand to promote the uptake of nanoparticles by M. smegmatis. Trehalose is a nonmammalian disaccharide sugar and can be abundant in free form and in glycoconjugates and can be found in cytosol and outer part of the mycobacterial cell envelope. Free trehalose is a major substituent part in cytosol. Its been known that 1.5%-3% of the M. smegmatis dry weight comprised with trehalose. It has been previously reported that trehalose is vital for M. smegmatis for both growth and to sustain viability at stationary phase.

Studies were conducted using phosphonate silane coated magnetic nanoparticles (SMNPs) and silica nanoparticles (SNPs) and fluorescent silica nanoparticles (FSNPs). The Tre conjugated nanoparticles were then treated with M. smegmatis strain mc² 155 and the uptake of nanoparticles by the bacteria cells were analyzed. As a control study, Glucose (Glc), Cyclodextrin(CD), and Maltoheptaose (G7) conjugated nanoparticles were used.

### Synthesis Of Magnetic Nanoparticles (MNPS) and Phosphonate-Silane Coated MNPS (SMNPS)

The synthesis of MNPs followed the previously reported procedure of Sun et al.⁴⁶ Iron (III) acetylacetonate (0.706 g, 2.0 mmol), 1,2-hexadecanediol (2.584 g, 10.0 mmol), oleic acid (2.239 mL, 6.0 mmol) and oleylamine (2.820 mL, 6.0 mmol) in dibenzyl ether (20 mL) were stirred under a blanket of nitrogen. The mixture was then heated to 200° C. for 2 h, followed by heating at 300° C. for 1 h. After cooling down to room temperature, ethanol (200-proof, 40 mL) was added and the mixture was centrifuged at 7000 rpm for 10 min. The black precipitate was re-dispersed in hexanes (30 mL) containing oleic acid (0.05 mL) and oleylamine (0.05 mL), and the mixture was centrifuged at 6000 rpm for 10 min. The precipitate was discarded, and the supernatant was collected and ethanol was added. After centrifugation, the precipitate was re-dispersed in hexanes.

To introduce silane coating on MNPs, a previously reported procedure of Ying et al.⁴⁷ was followed. MNPs (30 mg) were dispersed in toluene (15 mL), and a solution of phosphonate-silane in methanol (0.01 M, 3 mL) and a solution of TMAH in methanol (0.01 M, 3 mL) were added, and the mixture was stirred at 80° C. for 3 h (Scheme 1). After cooling down to room temperature, the mixture was centrifuged at 12,000 rpm for 30 min and the solid precipitate was re-dispersed in ethanol. This step was repeated for 3 times, and finally the nanoparticles were dispersed in ethanol (15 mL).

### Synthesis Of Pfpa-Functionalized SMNPS

2-(2′-(2″-(4-Azido-2,3,5,6-tetrafluorobenzamido)ethoxy)-ethoxy)ethyl dihydrogen phosphate (PFPA-phosphate) was synthesized following a previously reported procedure.⁴⁸ A solution of SMNP (2 mg/mL, 15 mL) in methanol was added to a solution of PFPA-phosphate in CHCl₃ (12 mg/mL, 3 mL), and the mixture was stirred at room temperature overnight followed by centrifugation at 12,000 rpm for 30 min. The supernatant was discarded, and the pellet was consecutively re-dispersed once in hexanes, twice in methanol and once in ethanol. After the final centrifugation, the supernatant was discarded and the resulting PFPA-functionalized MSNPs were re-dispersed in acetone/water (10 mL/20 mL).

### Synthesis of PFPA-Functionalized Dye-Doped Silica Nanoparticles (FSNPS)

FSNPs were synthesized by previously developed procedures. Florescein isothiocynante (FITC, 12 mM) was stirred overnight with aminopropyl trimethoxysilane (APTMS, 14 uL) in anhydrous ethanol at 42° C. to obtain the FITC-APTMS precursor. A 5 mL of FITC precursor was added to 34 mL of anhydrous ethanol with 2.0 mL of ammonia (6.25%) and 2.0 mL of Tetraethyl orthosilicate (TEOS). Mixture was stirred for 2 days to obtain FSNPs 50 nm. A solution of PFPA-silane in toluene (12.6 mM, 19 mL) was added to the FSNPs in anhydrous ethanol (43 mL, 2.3×10¹³ particles/mL, 23.4 mg/mL) and the mixture was stirred overnight followed by refluxing at 85° C. for 1 h (PFPA-silane⁵⁰ synthesized following previously-developed procedure). The mixture was centrifuged at 12,000 rpm for 18 min, and the precipitate was re-dispersed in ethanol for 3 times followed by in acetone for 3 times to give PFPA-functionalized FSNPs. The size of the PFPA functionalized FSNPs by DLS (55±10 nm) and by TEM (45±8 nm).

Synthesis of Carbohydrate-Functionalized SNPS, SMNPS

To a glass bottle containing an acetone solution of PFPA-FSNP (4 mg/mL, 1 mL) or PFPA-SMNP (1 mg/mL, 1 mL) was added an aqueous solution of carbohydrate Tre, Glc, G7 or CD (10.0 mg/mL, 200 μL). A 280 nm long-pass optical filter (Schott Glass Technologies Inc., Duryea, PA) was placed on top of the glass bottle, and the mixture was irradiated with a 450 W medium-pressure Hg lamp (3.2 mW/cm² measured by a 254 nm sensor) for 30 min while stirring. The resulting carbohydrate-conjugated nanoparticles were purified by centrifugation and re-dispersion in autoclaved Milli-Q water for 6 times followed by dialysis overnight.

### Uptake Of Carbohydrate-Conjugated Nanoparticles By M. Smegmatis

M. smegmatis strain mc2 155 was inoculated overnight in enriched Middlebrook 7H9 broth at 37° C. and shaken at 180 rpm. They were re-inoculated in fresh enriched Middlebrook 7H9 medium and grown until an OD₆₅₀ of 0.5 was attained (OD of 0.3 corresponds to ˜1.5×10⁸ bacteria cells). The bacteria cells (30 mL, ˜3×10⁹ bacteria cells) were then harvested, centrifuged at 5,000 rpm, and re-dispersed in pH 7.4 PBS buffer (5.0 mL). The carbohydrate-conjugated nanoparticles (˜1 mg/mL, 200 μL) were added to an aliquot of

M. smegmatis (500 μL, ˜3×10⁸ bacteria cells), and the mixture was incubated at 37° C. for 6 h while shaking at 180 rpm. The mixture was then centrifuged at 1,500 rpm for 10 minutes, and the supernatant containing nanoparticles was discarded. The pellet was then re-dispersed in autoclaved PBS. This centrifugation and redispersion step was repeated for 3 times.

### Nanoparticle Size Effect

A series of varying size of streptomycin conjugated silica nanoparticles (SNP-Str) can be used to target and effectively inhibit streptomycin resistant Escherichia coli (E. coli) bacteria lowering the minimum inhibitory concentration (MIC) of streptomycin upto 2 log folds. The diverse particle size affects inhibition and control. Silica nanoparticles were synthesized with an average of 80, 50 and 30 nm. Streptomycin was conjugated via photocoupling method through perfluorophenyl azide (PFPA) chemistry. The MIC for free streptomycin sulfate was recorded as a high 2.0 mg/mL for an engineered Str^{r} mutant E. coli ORN 208. Conjugating the streptomycin to the particles bought down the MIC from 2000 mg/mL to a low 19 μg/mL. The varying SNP sizes demonstrated an interesting variation in MIC from 161 μg/mL (SNP8O-Str), 63 μg/mL (SNP50-Str), 19 μg/mL (SNP30-Str).

It is reasonable to hypothesize that the more nanoparticles internalize into the bacteria the greater the effect of the antibiotic. Hence a higher antibacterial effect is seen with the smaller SNP30-Str's. The comparative difference of the streptomycin that can exert MIC is shown in FIG. 9 for the engineered Str^{r} mutant E. coli ORN 208 .

### Synthesis of Tricosa-O-Acetyl-A-Maltoheptaose (1)

200 mg of FeCl₃ was dissolved in 12.5 mL of Ac₂O and 4 g of β-cyclodextrin was slowly added in ice bath. The mixture was stirred for 2.5 h at room temperature followed by 3.5 h at 70° C. After cooling to room temperature, the red solution was poured into 500 mL of water. The precipitation was collected and wash with water and cold ethanol. White solid was obtained after 3 times recrystallization in ethanol.

### Synthesis of Docosa-O-Acetyl-A-Maltoheptaosyl Bromide (2)

3 mL of HBr-CH₃COOH solution was added into dry CH₂Cl₂ that contained 2 g of tricosa-O-acetyl-α-maltoheptaose and stirred for 3.5 h with ice bath. Then, the mixture was diluted to 50 mL with CH₂Cl₂ and washed with NaHCO₃ (10%, 20mL), water and saturated NaCl. Finally, white solid was collected after removing the solvent.

Synthesis of Docosa-O-Acetyl-A-Maltoheptaosylazide (3)

2 g of docosa-O-acetyl-α-maltoheptaosyl bromide was dissolved in 5 mL of dry THF. A solution of 3.5 mL of 1M TBAF in THF and 0.45 mL azidotrimethylsilane were added. The mixture was stirred overnight. 50 mL of CH₂Cl₂ was then added and the solution was washed with water. The CH₂Cl₂ phase was dried over Na₂SO₄ and the solvent was evaporated to yield the product.

### Synthesis of Maltoheptaosylazide (4)

2 g of docosa-O-acetyl-α-maltoheptaosylazide was added into 40 mL of 1:1 methanol:THF and 1 mL of 25% CH₃ONa in methanol. After 1 day, the white solid was centrifuged, and washed with THF and ethanol to afford maltoheptaosylazide.

### Synthesis of Acetylene-Terminated PLA (5)

L-lactide was purified by recrystallization in ethyl acetate. 4 g of L-lactide, 20 mg of Sn(Oct)₂ and 40 mg of 4-pentyn-1-ol were added into a flame-dried flask. The solution was purged with Ar and was heated to 120° C. for 3 h. After cooling to room temperature, the product was purified by dissolving in CH₂Cl₂ and precipitating in hexane three times.

### Synthesis of Maltoheptaose-PLA (6)

To 5 mL DMSO was added 200 mg of maltoheptaosylazide and 800 mg of acetylene-terminated PLA (5). The solution was bubbled with Ar for 1 h. 15 mg of CuSO₄ and 40 mg of sodium ascorbate were added, and the mixture was bubbled with Ar for another hour. The reaction was continued to stir for 2 days. The mixture was then poured into water and was then dialyzed against water for two days. A white powder was obtained after lyophilization.

### Preparation of Streptomycin-Loaded Maltoheptaose-PLA Micelles

An aqueous solution of streptomycin sulfate (120 μL, 16 mg/mL) was added into 4 mL of dichloromethane (DCM) that contained 100 mg of G7-PLA polymer. The mixture was then sonicated (150 W) for 30 seconds. The water/DCM emulsion was then injected into PVA water solution (20 mL, 0.1%, Mw: 30000) and was subjected to sonication for 2 min to form water/oil/water. DCM was removed by rotavapor. The resulting micelles were centrifuged and washed with water to remove free streptomycin followed by lyophilization.

### Theoretical Discussion

Although the theoretical description given herein is thought to be correct, the operation of the devices described and claimed herein does not depend upon the accuracy or validity of the theoretical description. That is, later theoretical developments that may explain the observed results on a basis different from the theory presented herein will not detract from the inventions described herein.

Any patent, patent application, patent application publication, journal article, book, published paper, or other publicly available material identified in the specification is hereby incorporated by reference herein in its entirety. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

While the present invention has been particularly shown and described with reference to the preferred mode as illustrated in the drawing, it will be understood by one skilled in the art that various changes in detail may be affected therein without departing from the spirit and scope of the invention as defined by the claims.

What is claimed is:

1. A functionalized nanoparticle, comprising:
a nanoparticle having a dimension in the range of units of nanometers to one hundred nanometers, and having an external surface;
a carbohydrate attached to the external surface of the nanoparticle, the carbohydrate having a chemical property that can induce a living cell to ingest the functionalized nanoparticle; and
a chemical attached to the external surface of the nanoparticle, the chemical known to have a biological effect on the living cell.

2. The functionalized nanoparticle of claim 1, wherein the nanoparticle is selected from the group of nanoparticles consisting of magnetic nanoparticles, silica-coated magnetic nanoparticles, silica nanoparticles, fluorescent silica nanoparticles, and silica-coated quantum dots.

3. The functionalized nanoparticle of claim 1, wherein the carbohydrate is selected from the group of carbohydrate consisting of D-trehalose, D-glucose, D-maltoheptaose, fructose, sucrose, and cyclodextran.

4. The functionalized nanoparticle of claim 1, wherein the living cell is selected from the group of cells consisting of a bacterium, a protozoan, a fungus, and a mammalian cell.

5. The functionalized nanoparticle of claim 1, wherein the biological effect is selected from the group of biological effects consisting of improving the health of a living cell, curing a diseased living cell and killing a diseased living cell.

6. The functionalized nanoparticle of claim 1, wherein the chemical is an antibiotic.

7. The functionalized nanoparticle of claim 6, wherein the antibiotic is streptomycin.

8. The functionalized nanoparticle of claim 6, wherein the antibiotic is a selected one of ampicillin, vancomycin, kanamycin, isoniazid, rifampicin, amikacin, rifabutin, ethambutol, capreomycin and pyrazinamide.

9. The functionalized nanoparticle of claim 6, wherein the antibiotic is a selected one of aminoglycosides, glycopeptides, macrolides, sulfonamides, statins, tetracyclines, peptide antibiotics, antimicrobial peptide, and quinolones.

10. The functionalized nanoparticle of claim 1, wherein the chemical is a cell penetrating peptide or a therapeutic siRNA.

11. A functionalized nanoparticle, comprising:
a nanoparticle having a dimension in the range of units of nanometers to one hundred nanometers, and having an internal surface and an external surface;
a carbohydrate attached to the external surface of the nanoparticle, the carbohydrate having a chemical property that can induce a living cell to ingest the functionalized nanoparticle; and
a chemical attached to the internal surface of the nanoparticle, the chemical known to have a biological effect on the living cell.
